(19) United States
(12) Patent Application Publication
(10) Pub. No.: US 2004/0224010 A1
(43) Pub. Date: Nov. 11, 2004

(54) Ophthalmic liposome compositions and uses thereof

(75) Inventors: Hans Hofland, Foster City, CA (US); Juiiet Bongianni, Sonoma, CA (US); Tobias Wheeler, Sebastopol, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: OPTIME Therapeutics, Inc., Petaluma, CA

(21) Appl. No.: 10/713,787

(22) Filed: Nov. 13, 2003

Related U.S. Application Data
(60) Provisional application No. 60/426,501, filed on Nov. 15, 2002.

(51) Int. Cl. A61K 9/127

(52) U.S. Cl. 424/450

(57) ABSTRACT

The present invention provides, inter alia, novel, liposome-based formulations for enhanced ophthalmic drug delivery. Such ophthalmic drug formulations are useful for treating ocular inflammation stemming from, for example, iritis, conjunctivitis, seasonal allergic conjunctivitis, acute and chronic endophthalmitis, anterior uveitis, uveitis associated with systemic diseases, posterior segment uveitis, chorioretinitis, pars planitis, masquerade syndromes including ocular lymphoma, pemphigoid, scleritis, keratitis, severe ocular allergy, corneal abrasion, or blood-aqueous barrier disruption, and for treating post-operative ocular inflammation, resulting from, for example, photorefractive keratectomy, cataract removal surgery, intraocular lens implantation or radial keratotomy.

### CROSS-REFERENCES TO RELATED APPLICATIONS

This application claims the benefit of U.S. Provisional Application No. 60/426,501, filed Nov. 15, 2002, which application is incorporated herein by reference for all purposes.

### BACKGROUND OF THE INVENTION

Topical application of ophthalmic drugs is not particularly efficient. After instillation of eyedrops, a substantial amount of the applied dose is rapidly cleared from the eye through blinking, nasolacrimal drainage, and tear production (Davies, Clin. Exp. Pharmacol. Physiol., 27(7):558-62 (2000)). This significantly reduces the therapeutic value of the drug. Furthermore, if the ultimate target is an intraocular structure, corneal penetration of a drug is paramount. The cornea is a formidable barrier composed of alternating hydrophobic-hydrophilic-hydrophobic layers (Ranade et al., Drug Delivery Systems, CRC Press (1996)). Therefore, while eyedrops may be useful for treating symptoms associated with exterior surfaces of the eye, they have little value in the treatment of intraocular disorders. Designing a drug or a drug delivery vehicle that can effectively penetrate the corneal barrier and deposit a therapeutic dose presents a significant challenge to the formulation scientist.

Numerous delivery methods have been developed in an effort to boost drug retention and ocular absorption of applied drug (Le Bourlais et al., J. Microencap., 14(4):457-467 (1997)). The least invasive of these methods include the use of drug-containing gels or micro- or nano-particulate suspensions. Generally speaking, such delivery vehicles are a significant improvement over eyedrops, but are not without undesirable side-effects. For example, gels have a tendency to cause blurred vision or sticking of the eyelids, while polymeric microparticulates can cause ocular irritation. Additionally, microparticulates have a tendency to settle in the dropper bottle and may be difficult to sterilize prior to packaging (Davies, Clin. Exp. Pharmacol. Physiol., 27(7):558-62 (2000)).

As such, what is needed in the art are drug delivery vehicles that can be used to deliver ophthalmic drugs to eye to treat various ophthalmic disorders. Quite surprisingly, the present invention provides such drug delivery vehicles.

### BRIEF SUMMARY OF THE INVENTION

The present invention provides lipid formulations, e.g., liposomal formulations, for delivering ophthalmic drugs to the eye. It has been found that by incorporating ophthalmic drugs into the lipid formulations of the present invention, a higher maximum concentration (Cmax) and longer drug residence time (T½) in ocular tissues can be achieved. Unlike topically applied solutions, the lipid formulations, e.g., liposomal formulations, can target and adhere to the corneal surface. Such delivery systems provide a drug reservoir at the ocular surface, which is not cleared as rapidly as topically applied eyedrops, and which is not as irritating as drug-containing ocular inserts or polymeric microparticulates. Such novel lipid formulations have multiple applications for a wide variety of topically applied ophthalmic drugs.

The lipid formulations of the present invention can be used, for example, for reducing inflammation due to seasonal or bacterial conjunctivitis, for reducing post-surgical pain and inflammation, to prevent or treat fingal or bacterial infections of the eye, to treat herpes ophthalmicus, to reduce intraocular pressure, or to treat endophthalmitis. Additionally, such lipid formulations can be used to deliver drugs prior to a routine eye examination or eye surgery.

### DETAILED DESCRIPTION OF THE INVENTION

As explained above, the present invention provides lipid-based (e.g., liposome-based) ophthalmic drug delivery vehicles that are superior to the current eye drop technology with respect to intraocular drug concentration and drug residence time. The lipid formulations of the present invention tenaciously coat the ocular surface, resulting in a drug reservoir within the eye. More particularly, the liposome-based drug delivery vehicles of the present invention have an increased ophthalmic residence time, ultimately increasing the amount of drug delivered, and thereby increasing the depth of drug penetration into the eye.

A. Lipid Formulations

In one aspect, the present invention provides a lipid formulation, the lipid formulation comprising: a lipid phase, the lipid phase comprising a neutral lipid and a member selected from the group consisting of a cationic lipid and a mucoadhesive compound; an aqueous phase; and a therapeutic agent. In a preferred embodiment, the lipid formulation is a liposome, a nanocapsule, a microparticle, a microsphere, a lipid complex, and the like. In a presently preferred embodiment, the lipid formulation is a liposome and the therapeutic agent is encapsulated in or associated with the liposome.

In one embodiment, the lipid phase comprises a neutral lipid as well as a cationic lipid or a mucoadhesive compound. Suitable neutral lipids include any of a number of lipid species which exist either in an uncharged or neutral zwitterionic form at physiological pH. Such neutral lipids include, but are not limited to, phospholipids, such as phosphatidylcholine, sphingomyelin, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine and dilinoleoylphosphatidylcholine. In a preferred embodiment, the neutral lipid is a phosphatidylcholine, such as Phospholipon 90H, Phospholipon 80H or a mixture thereof. In another preferred embodiment the phosopholipid includes, but is not limited to, phosphatidyl choline (PC), lyso-phophatidyl choline (l-PC), phosphatidyl serine (PS), phosphatidyl ehtanolamine (PE), phosphatidyl glycerol (PG), and phosphatidyl inisotol (PI). Suitable cationic lipids include those that carry a net positive charge at physiological pH. Such cationic lipids include, but are not limited to, N,N-dioleyl-N,N-dimethylammonium chloride (“DODAC”); N-(2,3-dioleyloxy)propyl-N,N-N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 3β-(N-(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethylammonium trifluoracetate (“DOSPA”), dioctadecylamidoglycyl carboxyspermine (“DOGS”), 1,2-dileoyl-sn-3-phosphoethanolamine (“DOPE”); N-(1,2-dimyristyloxyprop-3-yl)-N,N-dimethyl-N-hydroxyethyl ammonium bromide (“DMRIE”); stearylamine; dimethyldioctadecylammonium bromide; and 3B-[N′,N′-dimethylaminoethane)-carbamol. In a preferred embodiment, the cationic lipid is, for example, stearylamine, DC-Cholesterol, dimethyldioctadecylammonium bromide, or 3B-[N′,N′-dimethylaminoethane)-carbamol. Suitable mucoadhesive compounds include, but are not limited to, Carbopol 934 P, polyaxomers, carbomers and plant lectins.

In one embodiment, the aqueous phase includes, but is not limited to, sterile water sterile saline and sterile, isotonic aqueous solutions buffered in the pH range of about 6.5 to about 8.5 with, for example, sodium acetate, sodium phosphate, boric acid and the like. Other suitable pharmaceutical carriers are described in Remington's Pharmaceutical Sciences. In a preferred embodiment, the therapeutic agent is present in the aqueous phase.

In one embodiment, the lipid formulation further comprises a preservative, such as an antioxidant. Suitable preservatives/antioxidants include, but are not limited to, tocoperol (e.g., alpha-tocopherol), tocopherol derivatives, butylated hydroxyanisole and butylated hydroxytoluene.

In another embodiment, the lipid formulation further comprises a modifying agent including, but not limited to, cholesterol, stearylamine, cholesteryl hemisuccinate, phosphatidic acids, dicetyl phosphate and fatty acids.

In still another embodiment, the lipid formulation further comprises a wetting agent. Suitable wetting agents include, but are not limited to, polyoxyethylene, sorbitan monolaurate and stearate.

In still another embodiment, the lipid formulation further comprises a thickening agent. Suitable thickening agents include, but are not limited to, hydroxyethylcellulose, hydroxypropylmethylcellulose, methylcellulose, polyvinyl alcohol and polyvinylpyrrolidone.

In a preferred embodiment, the lipid formulation further comprises a preservative (e.g., antioxidant), a modifying agent, a wetting agent, a thickening agent or a combinaiton of any or all of the foregoing.

Suitable therapeutic agents include, but are not limited to, those agents set forth in Table 1.

TABLE 1
Ocular liposome formulations of active agents.

| Drug class | Exemplified by, but not limited to: |
|---|---|
| Antibiotics and Antivirals | Acyclovir (11-15) |
| | Dihydrostreptomycin (9) |
| | Fatty Acids |
| | Foscarnet (16-18) |
| | Gentamicin (10) |
| | Idoxuridine (7) |
| | Monoglycerides |
| | Penicillin G (8) |
| | Povidone-lodine (19-21) |
| Corticosteroids and Non-ster- | Dexamethasone sodium phosphate (24,25) |
| oidal | |
| Anti-inflammatory Agents | Dexamethasone, dexamethasone esters |
| | (23) |
| | Diclofenac |
| | Indoxole (8) |
| | Triamcinolone acetonide (22) |
| Mydriatic Agents | Atropine (31) |
| | Carbachol (32) |
| | Epinephrine (26) |
| | Pilocarpine (27-30) |
| | Tropicamide (33,34) |
| Local Anesthetics | Procaine and derivatives (35) |
| Antimitomic and Myectic | Cytrarabine (37) |
| Agents | Doxorubicin (36) |
| | Mitoxanthrone (38) |
| Immunosuppresive Agents | Cyclosporin A (39-41) |
| | Pacrolimus |
| Model/Other Drugs | DNA |
| | Inulin (26,42) |
| | Oligodeoxynucleotides (43,44) |
| | Prostaglandins |

In a preferred embodiment, the therapeutic agent is diclofenac and, in particular, sodium diclofenac.

A therapeutically effective amount of the therapeutic agent is delivered to the eye of the mammal. The term “therapeutically effective amount” refers to that amount of the therapeutic agent (e.g., diclofenac) which, when administered to a mammal in need thereof, is sufficient to effect treatment as, for example, an anti-inflammatory agent. The amount that constitutes a “therapeutically effective amount” will vary depending on the condition or disease and its severity, and the mammal to be treated, its weight, age, etc., but may be determined routinely by one of ordinary skill in the art with regard to contemporary knowledge and to this disclosure.

In addition, the term “treatment” as used herein covers any treatment of a disease in a mammal, particularly a human, and includes: (i) preventing the disease from occurring in a subject which may be predisposed to the disease but has not yet been diagnosed as having it; (ii) inhibiting the disease, i.e., arresting its development; or (iii) relieving the disease, i.e., causing regression of the disease.

The amount of drug to be included in the liposomal preparation is not, pre se, critical and can vary within wide limits depending upon the intended application and the lipid used. The formulation to be administered will, in any event, contain a quantity of sodium diclofenac in an amount effective to alleviate the symptoms of the subject being treated. The level of diclofenac sodium in the liposomal formulation of the invention can vary within the full range employed by those skilled in the art, e.g., from about 0.01 percent weight (wt %) to about 99.99 wt % sodium diclofenac, based on the total formulation weight. Preferably, diclofenac may be included in an amount of between about 0.01 to 10 wt % of the liposomal preparation. For products intended to be sold “over-the-counter,” a lower dosage is desired, preferably within the range of 0.01 to 1 wt %. On the other hand, for products intended to be sold “by-prescription-only,” a higher dosage is desired, preferably within the range of 0.1 to 10 wt %.

Generally, a daily dose of a 0.1% ophthalmic sodium diclofenac solution is from about one to five 50 mL drops per eye and most preferably from about three to four 50 mL drops per eye. Thus, the dosage range for each eye would be about 50 to 250 mg per day and most preferably 150 to 200 mg per day, assuming total absorption. The amount of sodium diclofenac administered will, of course, be dependent on the subject and disease state being treated, the severity of the affliction, the manner and schedule of administration and the judgment of the prescribing physician. Such use optimization is will within the ambit of those of ordinary skill in the art.

The preferred formulation is typically comprised of 0.01 to 10 wt % lipid phase and 99.99 to 90 wt % aqueous phase; more preferably, 0.1 to 1 wt % lipid phase and 99.9 to 99.0 wt % aqueous phase. A particularly preferred formulation is comprised of 0.1 wt % lipid phase and 99.9 wt % aqueous phase.

The lipid phase is typically comprised of 0.01 to 10 wt % phospholipids, 0.1 to 10 wt % modifying agents and 0.1 to 10 wt % antioxidant; more preferably, 0.1 to 1 wt % phospholipids, 0.01 to 1 wt % modifying agents and 1 to 5 wt % antioxidant.

B. Methods for Preparing the Lipid Formulations

Various methods of liposome and liposome-like preparations as potential drug carriers have been reviewed (see, e.g., U.S. Pat. Nos. 5,567,434, 5,552,157, 5,565,213, 5,738,868 and 5,795,587, each specifically incorporated herein by reference in its entirety).

Materials and procedures for forming liposomes are well known to those skilled in the art and will only be briefly described herein. Upon dispersion in an appropriate medium, a wide variety of phospholipids swell, hydrate and form multilamellar concentric bilayer vesicles of spherical geometry with layers of aqueous media separating the lipid bilayers. These systems are referred to as multilamellar liposomes or multilamellar lipid vesicles (MLVs) and have diameters within the range of 25 nm to 4 μm. These MLVs were first described by Bangham et al., J. Mol. Biol., 13:238-252 (1965). In general, lipids or lipophilic substances are dissolved in an organic solvent. When the solvent is removed, such as under vacuum by rotary evaporation, the lipid residue forms a thing film on the wall of the container. An aqueous solution that typically contains electrolytes or hydrophilic biologically active materials is then added to the container. Large MLVs are produced upon agitation. When smaller MLVs are desired, the larger vesicles are subjected to sonication or sequential filtrations through filters with decreasing pore size. There are also techniques by which MLVs can be reduced both in size and in number of lamellae, for example, by pressurized extrusion (Barenholz et al., FEBS Lett., 99:210-214 (1979)).

Liposomes can also take the form of unilamellar vesicles, which are prepared by more extensive sonication of MLVs, and consist of a single spherical lipid bilayer surrounding an aqueous solution. Unilamellar lipid vesicles (ULVs) can be small, having diameters within the range of 200-500 Å, while larger ULVs can have diameters within the range of 1000-10,000 Å. There are several well-known techniques for making unilamellar vesicles. In Papahadjopoulos et al., Biochim et Biophys Acta, 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri et al., Biochim et Biophys Acta, 298:1015-1019 (1973) and the ether injection technique of Deamer et al., Biochim et Biophys Acta, 443:629-634 (1976). These methods involve the rapid injection of an organic solution of lipids into an aqueous buffer solution, which results in the rapid formation of unilamellar liposomes. Another technique for making ULVs is taught by Weder et al. in “Liposome Technology”, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing an aqueous phospholipid dispersion of the agent and lipids.

In addition to MLVs and ULVs, there are also multivesicular liposomes (MVL). Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septa.

Mezei et al., U.S. Pat. No. 4,485,054, and Mezei, U.S Pat. No. 4,761,288, also describe methods of preparing lipid vesicles.

A comprehensive review of all the aforementioned lipid vesicles and methods for their preparation are described in “Liposome Technology” ed. G. Gregoriadis, CRC Press Inc., Boca Raton, Fla., Vol. I, II, & III (1984). This and the aforementioned references describing various lipid vesicles suitable for use in the invention are incorporated herein by reference.

In a presently preferred embodiment, the lipid formulations, e.g., liposomes, of the present invention are produced using the apparatus and method described in PCT Publication No. WO 00/29103, published on May 25, 2000, and incorporated herein by reference.

An apparatus is described therein that is useful for the continuous production of a composition of matter by in-line mixing. The apparatus comprises a first phase storage means capable of being maintained at a set temperature and a first pressurized transfer means for transferring the first phase from the storage means, along with an second phase storage means capable of being maintained at a set temperature and a second pressurized transfer means for transferring the second phase from the storage means. As described therein, the first phase is a lipid phase (optionally containing an active agent) and the second phase is an aqueous phase. The lipid phase storage means is capable of being maintained at a set temperature by a first temperature control means, typically within the range of about 20 to 75° C. Similarly, the aqueous phase storage means is capable of being maintained at a set temperature by a second temperature control means, typically within the range of about 20 to 75° C. The lipid phase and aqueous phase storage Means are equipped with a means for continuously replenishing the lipid and aqueous phases. In this manner, the storage means function as a temperature stabilization means such that the lipid and aqueous phases are continuously fed into the storage means, where the temperature of each phase is stabilized prior to introduction into pressurized transfer means that exits each respective storage vessel.

The apparatus also has a mixing device that comprises a first metering system for receiving the lipid phase from the first pressurized transfer means, a second metering system for receiving the aqueous phase from the second pressurized transfer means, a pre-mixing system for preparing a pre-mixed formulation, a third pressurized transfer means for transferring the lipid phase from the first metering system to a first inlet orifice in the pre-mixing system and a fourth pressurized transfer means for transferring the aqueous phase from the second metering system to a second inlet orifice in the pre-mixing system. The pre-mixing system comprises a pre-mixing chamber having a first and second inlet orifice. The pre-mixing system can further comprise a means for creating turbulence in the aqueous phase prior to entry into the pre-mixing chamber.

The apparatus also has a mixer such as a static mixer for preparing a mixed formulation comprising lipid vesicles, having a mixing chamber and an optional means for determining the optical properties of the mixed formulation, a fifth pressurized transfer means for transferring the pre-mixed formulation from the outlet orifice of the pre-mixing system to the mixing chamber or other suitable connection or fitting; and an optional means for applying ultrasonic energy to the pre-mixing system, the mixing chamber or both. In a preferred embodiment, the optical properties of the mixed formulations are measured, with the means for determining the optical properties of the mixed formulation being configured so as to control the first and second temperature control means and the first and second metering systems.

The apparatus and method of the invention provide for lipid phase and aqueous phase streams that are as pulse-less as possible and are maintained at a constant pressure. This is a achieved by the precise metering systems each of which is provided with a pump that operates under positive pressure and in such a manner so as to provide precise volumetric delivery.

The mixer is preferably a static mixer, such as a laminar division type inline mixer. The mixer may have a means for controlling the temperature of the mixing chamber, which is typically within the range of about 20 to 80° C. In addition, the mixer may also have a means for controlling the degree and rate of mixing within the mixing chamber. The mixing device of the apparatus may also have a means for controlling the temperature within the open space of the mixing device, which is also typically within the range of about 20 to 80° C.

The apparatus has a dispensing means for transferring the mixed formulation from the mixing chamber into a storage chamber. This apparatus is particularly useful for the production of lipid vesicles, and more particularly multilamellar lipid vesicles. The apparatus of the invention is readily evaluated as to its particular suitability for manufacturing lipid vesicles having a pre-specified composition and configuration.

In general, liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

C. Methods of Using the Lipid Formulations of the Present Invention to Treat Ophthalmic Disorders

The lipid formulations of the present invention can be used, for example, for reducing inflammation due to seasonal or bacterial conjunctivitis, for reducing post-surgical pain and inflammation, to prevent or treat fungal or bacterial infections of the eye, to treat herpes ophthalmicus, to reduce intraocular pressure, or to treat endophthalmitis. Additionally, such lipid formulations can be used to deliver drugs prior to a routine eye examination or eye surgery.

More particularly, in one embodiment, the present invention provides a method for treating an ophthalmic disorder in a mammal (e.g., a human), the method comprising administering to the eye of the mammal a therapeutically effect amount of a lipid formulation of the present invention comprising a lipid phase, an aqueous phase and a therapeutic agent, wherein the therapeutic agent is useful for treating the ophthalmic disorder. In one embodiment, the ophthalmic disorder is post-operative pain. In another embodiment, the ophthalmic disorder is ocular inflammation resulting from, e.g., iritis, conjunctivitis, seasonal allergic conjunctivitis, acute and chronic endophthalmitis, anterior uveitis, uveitis associated with systemic diseases, posterior segment uveitis, chorioretinitis, pars planitis, masquerade syndromes including ocular lymphoma, pemphigoid, scleritis, keratitis, severe ocular allergy, corneal abrasion and blood-aqueous barrier disruption. In yet another embodiment, the ophthalmic disorder is post-operative ocular inflammation resulting from, for example, photorefractive keratectomy, cataract removal surgery, intraocular lens implantation and radial keratotomy.

D. Modes of Administration

In employing the ophthalmic-liposome formulations of the present invention, any pharmaceutically acceptable mode of ocular administration can be used. Administration is preferably via a local route, for example, via intravitreal or subconjunctival injection or via ocular application in liquid, emulsion, suspension or aerosol dosage forms. The formulations of the present invention can also be administered in sustained or controlled release dosage forms, including depot injections, osmotic pumps and the like, for prolonged administration of the therapeutic agent at a predetermined rate, preferably in unit dosage forms suitable for single administration of precise dosages.

More particularly, the preferred method of administration is ocularly, which term is used to mean delivery of therapeutic agents through the surface of the eye, including the sclera, the cornea, the conjunctiva and the limbus. Ocular delivery can be accomplished by numerous means, for example, by topical application of a formulation such as an eye drop, by injection, or by means of an electrotransport drug delivery system.

### EXAMPLES

A. Preparation of Liposomal Drug Delivery Vehicles

Various biologically inactive, mucoadhesive compounds or cationic lipids can be added to the following basic formulation and screened for their ocular residenc time: Liposomes will be made up in 0.06 M boric acid buffer (pH 7.2) using 0.1% (w/v) phosphatidyl choline (Phospholipon 90-H), 0.1% (w/v) Sodium diclofenac, and either 0.0025% (v/v) benzalkonium chloride or 0.05% (w/v) chlorobutanol as a preservative. The ratio of cationic lipid to neutral phosphatidyl choline can be varied, and cationic lipids included in the liposomes to be tested include, but are not limited to, stearylamine, DC-Cholesterol, dimethyldioctadecylammonium bromide, or 3B-[N′,N′-dimethylaminoethane)-carbamol. Alternatively, mucoadhesive compounds (examples: Carbopol 934 P, Polyaxomers, Carbomers, or plant lectins) can be incorporated into the liposomes of the present invention.

Liposomes can be prepared by heating the lipid-containing, aqueous solutions to 65° C. for 10 minutes prior to mixing. The mixture is briefly homogenized and sonicated at 50° C. in a water bath sonicator to resize liposomes. Each solution is then filter sterilized by passage through a 0.2 micrometer syringe filter.

The physical and chemical characteristics of each formulation can be evaluated. The particle size can be measured over time using a Coulter N4 particle sizer. Drug encapsulation efficency of individual formulations can be determined using a gel filtration-spin column fractionation method (Fry et al., Anal. Biochem., 90:809 (1978)). This technique separates liposomes from unencapsulated diclofenac. Fractions containing liposomes can be identified using the Barlett phosphorus assay (Bartlett, J. Biol. Chem., 234:466 (1959)), and analysis of concentration and disposition of diclofenac in all fractions can be determined using HPLC/UV-Vis. The HPLC method includes a stationary phase of a C18 column (4.6 mm I.D.×250 mm) and a mobile phase of acetonitrile: 0.1% phopshoric acid (1:1). The flow rate used is 1 mL/min, and diclofenac is detected using a U/Vis detector set at 280 nm.

B. Ex Vivo Assay for Measuring Liposome Adhesion and Drug Absorption

Liposomal formulations that are physically and chemically stable, sterile, and that efficiently encapsulate the drug can be furthered screened for ex vivo drug absorption studies. Such experiments identify those formulations that boost drug absorption in the cornea over a defined period of time.

Such ex vivo studies are performed on bovine corneas using the method of Le Bourlais (Le Bouralais et al., Prog. Re. Eye Res., 17(1):33-58 (1992)). Excised corneas are placed in a concave aluminum cup, covered with a glass dish to prevent drying, and pre-equilibrated in an incubator at 25° C. for approximately 30 minutes. The liposome formulations (containing 50 micrograms of diclofenac) is applied directly onto the corneal surface. The formulation is allowed to stay in contact with the corneal surface for a defined period of time (30 seconds, 1 minute, 5 minutes, 10 minutes, 30 minutes, 1 hour, 3 hours).

After incubation, the cornea is rinsed with 50 mL of water, and a 5 mm diameter corneal button is removed from the cornea using a glass micropipette. This corneal button is digested in 0.5 mL buffered collagenase (1 mg/mL). One milliter of 0.05 M KH₂PO₄ (pH 3) is added to this digested corneal sample, vortexed and centrifuged at 3000 rpm for 10 minutes. The supernatant is removed and extracted twice with 3 mL ethyl accetate: n-hexane (1:1). The organic phase is removed, dried under nitrogen gas at 40° C., and resuspended in 100 microliters of mobile phase (acetonitrile: 0.1% phosphoric acid (1:1). Fifty microliters of this extract is analyzed for diclofenac content using the HPLC technique outlined in the previous experimental methods section. All time-points are carried out in duplicate, and three assays are carried out per cornea in order to account for any variation in applied dose.

This ex vivo model provides insight into the extent of liposome adhesion and the increase of drug absorption into corneal tissue. In doing so, it has been found that the mucoadhesive- and cationic lipid-containing liposome formulations of the present invention increase the retention time and the corneal drug concentration over that observed with neutral liposome formulations.

C. Assay for Measuring In Vivo Pharmacokinetics

The eye is an extremely complex organ, and it is difficult to assess the fate of an applied drug dose outside of using an in vivo model. Although the above ex vivo experiments are useful to determine the relative improvement of corneal adhesion of the liposome and absorption of a drug, such experiments do not take into account issues such as the blink reflex, drainage, irritation (toxicity). To be able to accurately determine how drug retention and absorption are affected by the inclusion of mucoadhesive compounds or cationic lipids, the liposome formulations of the present invention can be tested using the following in vivo model.

In vivo studies are carried out using male, Japanese white rabbits. A 50 μL volume of liposome formulation or Voltaren™ ophthalmic (commercially available diclofenac solution which will be used as a positive control) is placed into the inferior conjunctival sac. At 30 minutes, 1, 2, 4, 8, 12, and 24 hours following drug application, rabbits are euthanized (two per timepoint) by injecting 5 mL Nembutal (5% pentobarbital) intravenously into the ear vein. Eyes are rinsed well with saline, and the aqueous humor is collected via aspiration with a 1 mL syringe. The eyes are removed intact and the bulbar conjunctiva are removed from each eye, rinsed with saline and dried by blotting. The eyes are frozen using liquid nitrogen and bisected into the anterior and posterior segments using a razor. From the anterior segment, the cornea and the iris-ciliary body are removed, rinsed with saline and blotted. The vitreous humor, retina, and choroid are collected from the posterior segment. All samples are weighed and suspended in homogenization buffer. For cornea, bulbar conjunctiva, iris/ciliary body, and retina/choroid, 1 mL buffer (containing 500 mg/mL flurbiprofen as an internal standard) is added. For vitreous humor, 1 mL buffer (0.5 μL/mL flurbiprofen) is added. For aqueous humor, 100 μL buffer (5 μg/mL flurbiprofen) is added. Samples are then be homogenized using a blender type homogenizer, followed by a glass mortar/teflon pestle assembly. Following homogenization, 0.05 M KH₂PO₄ (pH 3) is added to each sample. One milliter is added to the aqueous humor sample, followed by two extractions with 3 mL ethyl acetate/n-hexane (1:1). Three milliliters of 0.05 M KH₂PO₄ is added to the vitreous humor sample, followed by centrifugation for 10 minutes at 3000 rpm. The supernatant is extracted twice with 3 mL ethyl acetate/n-hexane (1:1). The homogenate from the cornea, bulbar conjunctiva, iris/ciliary body, and retina/choroid is separated by centrifugation at 3000 rpm for 10 minutes. The supernatent is removed, and 1 mL of 0.05 M KH₂PO₄ is added, followed by two, 3 mL extractions with ethyl acetate/n-hexane (1:1). The organic phase from each extraction is dried under nitrogen gas at 40° C., and re-dissolved in 200 μL of mobile phase (acetonitrile/0.1% phosphoric acid (1:1). The diclofenac concentration in each ocular tissue is determined by running the samples using the HPLC/UV-Vis detection method, as described in connection with the ex vivo assay method.

Using the foregoing in vivo assay, it has been found that there is an initial increase in the diclofenac concentration in the cornea, aqueous humor, iris-ciliary body, and bulbar conjunctiva. However, unlike the conventional phosphatidylcholine-based liposomes, the formulations containing mucoadhesives and cationic lipids have greater increases in diclofenac concentration in all ocular tissues, as well as an increased intraocular residence time. Additionally, there is an increase in diclofenac in those ocular tissues in which diclofenac was not detected previously (i.e., the retina/choroid and vitreous humor).

D. Screening of the Liposomal Formulations

A number of phosphotidylcholine-based liposome formulations were created using diclofenac as a model drug. All formulations met the following, stringent criteria: they were produced without the use of an organic solvent, they were filter sterilized, they contained an antimicrobial agent, and they were stable with respect to particle size and did not contain any crystals or precipitates over a long period of time (at least 2 months). All formulations contained 0.1% w/v diclofenac (equal to the commercially available product Voltaren® ophthalmic) and an extremely small amount of lipid (0.1-0.3% w/v phosphatidylcholine), thus making the production of such ophthalmic formulations relatively inexpensive.

The most stable and pharmaceutically elegant of these formulations was chosen to be tested in vivo using the above-described rabbit eye model. During testing, a single application of liposome suspension containing 50 μg diclofenac or a commericially available solution (Voltaren®) containing 50 μg diclofenac was administered to the eyes of male, Japanese white rabbits. The rabbits were sacrificed at certain time intervals. The ocular tissues were dissected and analyzed for diclofenac using HPLC/UV-Vis spectrophotometry. Maximum concentrations of diclofenac (Cmax), peak areas (AUC), and half life of the drug (T ½) in a variety of ocular tissues is presented for both the liposome-based diclofenac ophthalmic and the Voltaren®.

The data indicate that the liposome-based diclofenac formulation boosts diclofenac concentrations substantially over levels achieved with the aqueous solution of diclofenac (Voltaren®). In ocular tissues, the liposome-based diclofenac formulation produced diclofenac levels anywhere from 2 fold greater in the iris-ciliary body to 3.2 fold greater in the cornea, over Voltaren®. On the other hand, the half life of diclofenac in all ocular tissues tested did not vary significantly between the liposome-based formulation and Voltaren®. Also, diclofenac was not detected in the retina/choroid or the aqueous humor with either of the formulations (see, Table I).

TABLE 1
Diclofenac Deposition in Ocular Tissues

| | | | | Liposome-encapsulated | | | |
|---|---|---|---|---|---|---|---|
| Ocular | Voltaren Ophthalmic (0.1%) ® | | | diclofenac (0.1%) | | | Fold |
| tissue | Cₘₐₓ | AUC | T_{1/2} | C_{mac} | AUC | T_{1/2} | increase |
| Aqueous | 0.38 | 1.84 | 3.38 h | 0.97 | 3.50 | 3.33 h | 2.6x |
| humor | μg/mL | μg · h/mL | | μg/mL | μg · h/mL | | |
| Cornea | 6.51 μg/g | 25.39 | 1.62 h | 20.5 μg/g | 59.51 | 2.17 h | 3.2x |
| | | μg · h/g | | | μg · h/g | | |
| Bulbar | 3.08 μg/g | 23.37 | 13.13 h | 7.09 μg/g | 28.50 | 9.49 h | 2.3x |
| conjuctiva | | μg · h/g | | | μg · h/g | | |
| Iris-ciliary | 0.56 μg/g | 1.91 | 2.18 h | 1.19 μg/g | 3.50 | 1.94 h | 2.1x |
| body | | μg · h/g | | | μg · h/g | | |

E. Other Exemplar Liposomal Formulations of the Present Invention

Formulation 1: A formulation containing 0.1% sodium diclofenac; 0.1% PHN-90H phospholipids; 0.1% benzethonium chloride; in 99.8% sterile saline (0.9% NaCl) was prepared as follows. Sodium diclofenac (pharmacopoeal grade), benzethonium chloride and PHN-90H phospholipids (American Lecithin Co., Atlanta, Ga.) are added to sterile saline solution. The mixture is heated to approximately 65° C. and then homogenized in a tissue homogenizer. Following this step, the mixture is sonicated at approximately 55° C. in a bath sonicator for 5 to 10 minutes to size the liposomes.

Formulation 2: A formulation containing 0.1% sodium diclofenac; 0.1% PHN-90H phospholipids; 0.5% chlorobutanol; in 99.3% sterile, isotonic boric acid buffer at pH 7.4 was prepared as follows. Sodium diclofenac (pharmacopoeal grade), chlorobutanol and PHN-90H phospholipids (American Lecithin Co., Atlanta, Ga.) are added to the isotonic boric acid buffer at pH 7.4. The mixture is heated to approximately 65° C. and then homogenized in a tissue homogenizer. Following this step, the mixture is sonicated at approximately 55° C. in a bath sonicator for 5 to 10 minutes to size the liposomes.

Formulation 3: A formulation containing 0.1% sodium diclofenac; 0.099% PHN-90H phospholipids; 0.001% stearylamine; 0.01% benzethonium chloride; in 99.89% sterile, isotonic boric acid buffer at pH 7.4 was prepared as follows. Sodium diclofenac (pharmacopoeal grade), stearylamine, benzethonium chloride and PHN-90H phospholipids (American Lecithin Co., Atlanta, Ga.) are added to the isotonic boric acid buffer at pH 7.4. The mixture is heated to approximately 65° C. and then homogenized in a tissue homogenizer. Following this step, the mixture is sonicated at approximately 55° C. in a bath sonicator for 5 to 10 minutes to size the liposomes.

What is claimed is:

1. A lipid formulation, said lipid formulation comprising:
a lipid phase, said lipid phase comprising a neutral lipid and a member selected from the group consisting of cationic lipids and mucoadhesive compounds;
an aqueous phase; and
a therapeutic agent.

2. A lipid formulation in accordance with claim 1, wherein said neutral lipid is a phospholipid.

3. A lipid formulation in accordance with claim 2, wherein said phospholipid is a soybean oil-based phospholipid.

4. A lipid formulation in accordance with claim 2, wherein said phospholipid is a member selected from the group consisting of phosphatidylglycerols (PG), phosphatidylethanolamines (PE), phosphatidylserines (PS) and hydrogenated phosphatidylcholines (PC).

5. A lipid formulation in accordance with claim 4, wherein said phospholipid is a phosphatidylcholine.

6. A lipid formulation in accordance with claim 5, wherein said phosphatidylcholine is a member selected from the group consisting of Phospholipon 90H, Phospholipon 80H and mixtures thereof.

7. A lipid formulation in accordance with claim 1, wherein said lipid phase comprises a cationic lipid.

8. A lipid formulation in accordance with claim 7, wherein said cationic lipid is a member of the group consisting of stearylamine, DC-Cholesterol, dimethyldioctadecylammonium bromide, or 3B-[N′,N′-dimethylaminoethane)-carbamol.

9. A lipid formulation in accordance with claim 1, wherein said lipid phase comprises a mucoadhesive compound.

10. A lipid formulation in accordance with claim 9, wherein said mucoadhesive compound is a member of the group consisting of Carbopol 934 P, polyaxomers, carbomers and plant lectins.

11. A lipid formulation in accordance with claim 1, wherein said aqueous phase is a member selected from the group consisting of sterile water, sterile saline and sterile, isotonic aqueous buffer solutions.

12. A lipid formulation in accordance with claim 11, wherein said aqueous phase is a sterile, isotonic aqueous solution buffered with borates, acetates, bicarbonates or phosphates in the pH range of 7.0 to 7.8.

13. A lipid formulation in accordance with claim 1, wherein said lipid formulation comprises about 0.001 to about 10.000 wt % of said lipid phase and about 90.000 wt % to about 99.999 wt % of said aqueous phase.

14. A lipid formulation in accordance with claim 1, wherein said lipid formulation comprises about 0.1 wt % of said lipid phase and about 99.0 wt % of said aqueous phase.

15. A lipid formulation in accordance with claim 1, wherein said therapeutic agent is present in said aqueous phase.

16. A lipid formulation in accordance with claim 1, wherein a therapeutically effective amount of said therapeutic agent is present in said lipid formulation.

17. A lipid formulation in accordance with claim 1, wherein said lipid formulation is a liposome.

18. A lipid formulation in accordance with claim 1, further comprising a preservative.

19. A lipid formulation in accordance with claim 18, wherein said preservative is an antioxidant.

20. A lipid formulation in accordance with claim 19, wherein said antioxidant is a member selected from the group consisting of tocoperol, tocopherol derivatives, butylated hydroxyanisole and butylated hydroxytoluene.

21. A lipid formulation in accordance with claim 18, wherein said preservative is an anti-microbial agent selected from the group consisting of benzalkonium chloride, benzethonium chloride, chlorobutanol, phenylethyl alcohol and cetyl pyridinium chloride.

22. A lipid formulation in accordance with claim 21, wherein said anti-microbial agent is chlorobutanol.

23. A lipid formulation in accordance with claim 1, further comprising a modifying agent selected from the group consisting of cholesterol, stearylamine, cholesteryl hemisuccinate, phosphatidic acids, dicetyl phosphate and fatty acids.

24. A lipid formulation in accordance with claim 1, further comprising a wetting agent.

25. A lipid formulation in accordance with claim 24, wherein said wetting agent is a member selected from the group consisting of polyoxyethylene, sorbitan monolaurate and stearate.

26. A lipid formulation in accordance with claim 1, further comprising a thickening agent.

27. A lipid formulation in accordance with claim 26, wherein said thickening agent is a member selected from the group consisting of hydroxyethylcellulose, hydroxypropylmethylcellulose, methylcellulose, polyvinyl alcohol and polyvinylpyrrolidone.

28. A lipid formulation in accordance with claim 1, wherein said therapeutic agent is a non-steroidal anti-inflammatory drug (NSAID).

29. A lipid formulation in accordance with claim 30, wherein said NSAID is a member selected from the group consisting ketoprofen, flurbiprofen, ibuprofen, diclofenac, ketorolac, nepafenac, amfenac and suprofen.

30. A lipid formulation in accordance with claim 30, wherein said NSAID is diclofenac.

31. A method for treating an ophthalmic disorder in a mammal, said method comprising administering to the eye of said mammal a lipid formulation in accordance with claim 1, wherein said therapeutic agent in said lipid formulation is useful for treating said ophthalmic disorder.

32. The method in accordance with claim 31, wherein said ophthalmic disorder is post-operative pain.

33. The method in accordance with claim 31, wherein said ophthalmic disorder is ocular inflammation.

34. The method in accordance with claim 33, wherein said ocular inflammation results from a member selected from the group consisting of iritis, conjunctivitis, seasonal allergic conjunctivitis, acute and chronic endophthalmitis, anterior uveitis, uveitis associated with systemic diseases, posterior segment uveitis, chorioretinitis, pars planitis, masquerade syndromes including ocular lymphoma, pemphigoid, scleritis, keratitis, severe ocular allergy, comeal abrasion and blood-aqueous barrier disruption.

35. The method in accordance with claim 31, wherein said ophthalmic disorder is post-operative ocular inflammation.

36. The method in accordance with claim 35, wherein said post-operative ocular inflammation results from a member selected from the group consisting of photorefractive keratectomy, cataract removal surgery, intraocular lens implantation and radial keratotomy.

37. The method in accordance with claim 31, wherein said ophthalmic disorder is a fingal or bacterial infection.

38. The method in accordance with claim 31, wherein said ophthalmic disorder is herpes ophthalmicus.

39. The method in accordance with claim 31, wherein said ophthalmic disorder is endophthalmitis.

40. The method in accordance with claim 31, wherein said ophthalmic disorder is intraocular pressure.

41. The method in accordance with claim 31, wherein said therapeutic agent is diclofenac.

42. The method in accordance with claim 41, wherein said diclofenac is diclofenac sodium.

43. A method for treating or preventing ocular inflammation, paracentesis-induced miosis, cystoid macular edema and mydriasis, said method comprising administering a therapeutically effective amount of one or more non-steroidal anti-inflammatory drugs encapsulated or contained within a liposome formulation, said liposome formulation comprising 0.001 to 10.000 wt % lipid phase, and 90.000 to 99.999 wt % aqueous phase.

44. The method in accordance with claim 43, wherein said liposome formulation is applied topically, resulting in the transcorneal or transscleral passage or introduction of one or more non-steroidal anti-inflammatory drugs into the eye.

45. The method in accordance with claim 43, wherein said lipid phase comprises 0.0 to 90.0 wt % of one or more active agents, 10.0 to 100.0 wt % phospholipid, 0.0 to 20.0 wt % antioxidant, and 0.0 to 20% modifying agents; and said aqueous phase comprises 0.0 to 10.0 wt % one or more active agents, 0.0 to 5.0 wt % anti-microbial preservative, and 90.0 to 100.0 wt % aqueous solution.

46. The method in accordance with claim 45, wherein said active agent(s) are non-steroidal anti-inflammatory drugs.

47. The method in accordance with claim 46, wherein said non-steroidal anti-inflammatory drugs are selected from the group consisting of ketoprofen, flurbiprofen, ibuprofen, diclofenac, ketorolac, nepafenac, amfenac and suprofen.

48. The method in accordance with claim 47, werein said non-steroidal anti-inflammatory drug is diclofenac.

49. The method in accordance with claim 43, wherein said ocular inflammation is a symptom of iritis, conjunctivitis, seasonal allergic conjunctivitis, post-operative inflammation, acute and chronic endophthalmitis, anterior uveitis, uveitis associated with systemic diseases, posterior segment uveitis, chorioretinitis, pars planitis, masquerade syndromes including ocular lymphoma, pemphigoid, scleritis, keratitis, severe ocular allergy, corneal abrasion, blood-aqueous barrier disruption or ocular trauma.

50. The method in accordance with claim 49, wherein said post-operative inflammation is caused by photorefractive keratectomy, cataract removal surgery, intraocular lens implantation or radial keratotomy.

51. A liposome formulation comprising: a therapeutic agent; 0.001 to 10.000 wt % of a lipid phase; and 90.000 to 99.999 wt % of an aqueous phase.

52. The liposome formulation in accordance with claim 51, wherein said lipid phase comprises a phospholipid.
